(19) United States
(12) Patent Application Publication
(10) Pub. No.: US 2009/0291996 A1
(43) Pub. Date: Nov. 26, 2009

(54) Caspofungin free of caspofungin Co

(76) Inventors: Ferenc Korodi, Debrecen (HU); Piroska Kovacs, Debrecen (HU); Andrea Csorvasi, Debrecen (HU); Gabor Nagy, Debrecen (HU)

Correspondence Address: KENYON & KENYON LLP, ONE BROADWAY, NEW YORK, NY, 10004, US

(21) Appl. No.: 12/454,776

(22) Filed: May 21, 2009

Related U.S. Application Data
(60) Provisional application No. 61/128,528, filed on May 21, 2008.
(60) Provisional application No. 61/130,845, filed on Jun. 3, 2008.
(60) Provisional application No. 61/133,319, filed on Jun. 26, 2008.

(51) Int. Cl. A61K 31/40; C07D 487/22; G01N 33/00; A61P 31/04

(52) U.S. Cl. 514/410; 540/461; 436/98

(57) ABSTRACT

The present invention provides Caspofungin and salts thereof substantially free of Caspofungin C₀ and salts thereof. The present invention also provides processes for the preparation of said Caspofungin and salts thereof and processes for the determination of the amount of Caspofungin C₀ and salts thereof present in Caspofungin and salts thereof. The present invention further provides pharmaceutical compositions comprising said Caspofungin and salts thereof.

### CROSS REFERENCE TO RELATED APPLICATIONS

The present invention claims the benefit of the following U.S. Provisional Patent Application Nos. 61/128,528, filed May 21, 2008; 61/130,845, filed Jun. 3, 2008; and 61/133,319, filed Jun. 26, 2008. The contents of these applications are incorporated herein by reference.

### FIELD OF INVENTION

The present invention relates to caspofungin free of caspofungin C₀, methods for preparation thereof and pharmaceutical compositions thereof.

### BACKGROUND OF THE INVENTION

Caspofungin, 1-[(4R,5S)-5-[(2-Aminoethyl)amino]-N2-(10,12-dimethyl-1-oxotetradecyl)-4-hydroxy-L-ornithine]-5-[(3R)-3-hydroxy-L-ornithine]-pneumocandin B₀, of the following formula

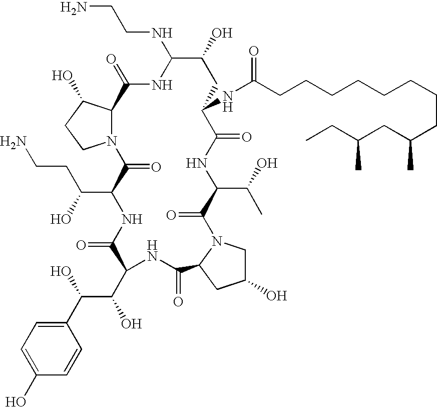

is a macrocyclic lipopeptide from the echinocandin family, which is known to be useful in treating systemic fungal infections, especially those caused by Candida, Aspergillus, Histoplasma, Coccidioides and Blastomyces. They have also been found useful for the treatment and prevention of infections caused by Pneumocystis carinii which are often found in immunocompromised patients such as those with AIDS. It is administrated as a diacetate salt as an intravenous infusion and sold under the trade name Cancidas® by Merck & Co., Inc.

Caspofungin is a semi-synthetic product that can be prepared from pneumocandin B₀, a natural product obtained from sources such as fermentation reactions, having the following formula:

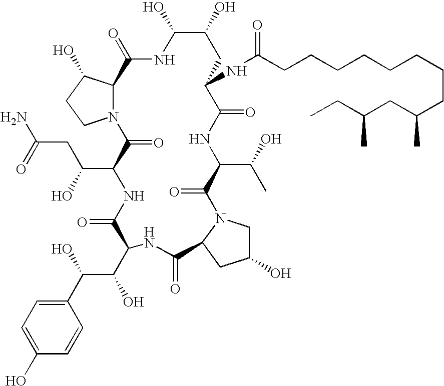

The preparation of pneumocandin B₀ is disclosed in several publications such as U.S. Pat. No. 5,194,377 and U.S. Pat. No. 5,202,309. The Journal of Antibiotics, 45, 1853, (1992) also describes the isolation of pneumocandin B₀ contaminated with a structurally related compound, named pneumocandin C₀, of the following formula:

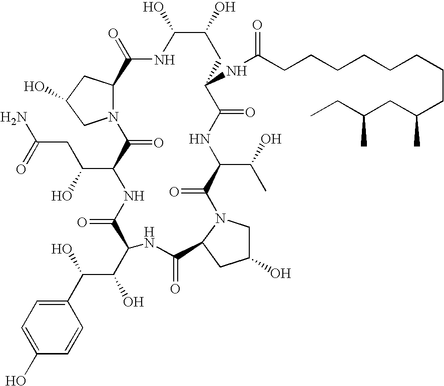

Caspofungin and its pharmaceutical acceptable salts are known under the INN (International Nonproprietary Name) caspofungin to be useful in treating fungal infections (see Merck Index, 13th edition, monograph no. 1899).

U.S. Pat. No. 5,378,804 describes caspofungin.

U.S. Pat. Nos. 5,552,521 and 5,936,062 describes synthetic approaches to prepare caspofungin. Caspofungin is also disclosed in EP2049142, US2009075870, and US2008319162

Like any synthetic compound, caspofungin may contain extraneous compounds or impurities. Impurities in caspofungin, or any active pharmaceutical ingredient (“API”), are undesirable and, in extreme cases, might even be harmful to a patient being treated with a dosage form containing the API.

The purity of an API produced in a manufacturing process is critical for commercialization. The U.S. Food and Drug Administration (“FDA”) requires that process impurities be maintained below set limits. For example, in its ICH Q7A guidance for API manufacturers, the FDA specifies the quality of raw materials that may be used, as well as acceptable process conditions, such as temperature, pressure, time, and stoichiometric ratios, including purification steps, such as crystallization, distillation, and liquid-liquid extraction. See ICH Good Manufacturing Practice Guide for Active Pharmaceutical Ingredients, Q7A, Current Step 4 Version (Nov. 10, 2000).

The product of a chemical reaction is rarely a single compound with sufficient purity to comply with pharmaceutical standards. Side products and by-products of the reaction and adjunct reagents used in the reaction will, in most cases, also be present in the product. At certain stages during processing of an API, it must be analyzed for purity, typically, by high performance liquid chromatography (“HPLC”) or thin-layer chromatography (“TLC”), to determine if it is suitable for continued processing and, ultimately, for use in a pharmaceutical product. The FDA requires that an API is as free of impurities as possible, so that it is as safe as possible for clinical use. For example, the FDA recommends that the amounts of some impurities be limited to less than 0.1 percent. See ICH Good Manufacturing Practice Guide for Active Pharmaceutical Ingredients, Q7A, Current Step 4 Version (Nov. 10, 2000).

Generally, side products, by-products, and adjunct reagents (collectively “impurities”) are identified spectroscopically and/or with another physical method, and then associated with a peak position, such as that in a chromatogram, or a spot on a TLC plate. See Strobel, H. A., et al., CHEMICAL INSTRUMENTATION: A SYSTEMATIC APPROACH, 953, 3d ed. (Wiley & Sons, New York 1989). Once a particular impurity has been associated with a peak position, the impurity can be identified in a sample by its relative position in the chromatogram, where the position in the chromatogram is measured in minutes between injection of the sample on the column and elution of the impurity through the detector. The relative position in the chromatogram is known as the “retention time.”

As known by those skilled in the art, the management of process impurities is greatly enhanced by understanding their chemical structures and synthetic pathways and by identifying the parameters that influence the amount of impurities in the final product.

The present invention thus addresses the need in the art for managing impurities in caspofungin and salts thereof, especially caspofungin C₀ and salts thereof, thus providing caspofungin and salts thereof free of caspofungin C₀ and salts thereof, and means for preparation thereof.

### SUMMARY OF THE INVENTION

In one embodiment, the present invention provides caspofungin and salts thereof of the following formula (n=0-3)

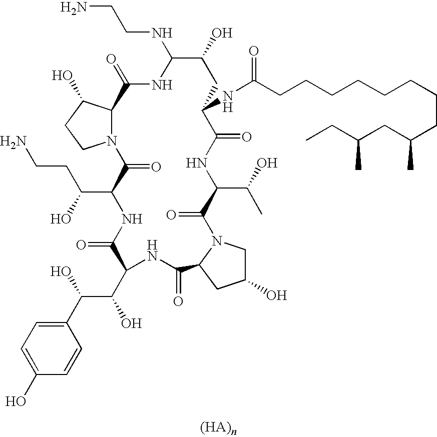

containing about 0.25% weight by HPLC or less of caspofungin C₀ and salts thereof of the following formula (n=0-3):

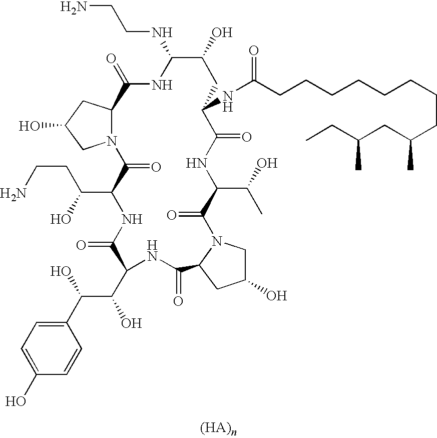

Preferably, the caspofungin is a diacetate salt (i.e. HA is acetic acid and n=2).

In another embodiment, the present invention provides a process for preparing caspofungin and salts thereof containing about 0.25% weight by HPLC or less of caspofungin C₀ and salts thereof from an initial sample of pneumocandin B₀ comprising:
a) analyzing the level of pneumocandin C₀ of the following formula:

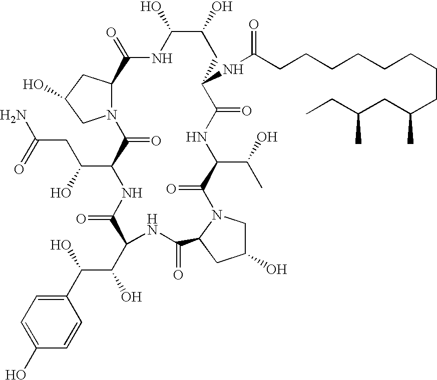

present in at least one initial sample of pneumocandin B₀ of the following formula:

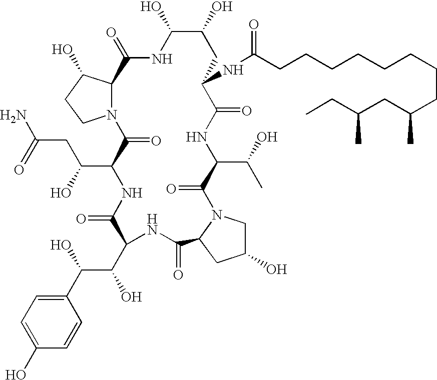

at least at a single stage during the synthesis process to control the amount of said caspofungin C₀ and salts thereof being present in the prepared caspofungin and salts thereof;
b) selecting as initial sample of pneumocandin B₀ that has no more than about 0.54% weight by HPLC of pneumocandin C₀, and
c) preparing caspofungin and salts thereof containing about 0.25% weight by HPLC or less of caspofungin C₀ and salts thereof from the said initial sample of pneumocandin B₀.

In another embodiment, the present invention provides a process for measuring the level of caspofungin C₀ and salts thereof in a sample of caspofungin and salts comprising the steps:
a) combining a sample comprising of caspofungin or salt thereof in a mixture of ammonium phosphate buffer and methanol to obtain a solution;
b) injecting the solution to a silica gel based polyamine HPLC (hydrophilic interaction chromatography) column;
c) eluting the sample from the column using an eluent of a mixture of acetonitrile: isopropanol and ammonium phosphate buffer; and
d) measuring the content of Caspofungin C₀ using a UV detector.

In yet another embodiment, the present invention provides a pharmaceutical composition comprising caspofungin and salts thereof containing about 0.25% weight by HPLC or less of caspofungin C₀ and salts thereof and at least one pharmaceutically acceptable excipient.

In another embodiment, the present invention provides the use of caspofungin and salts thereof containing about 0.25% weight by HPLC or less of Caspofungin C₀ and salts thereof in the manufacture of a pharmaceutical composition for the treatment of systemic fungal infections caused by Candida, Aspergillus, Histoplasma, Coccidioides and Blastomyces.

In another embodiment, the present invention provides the use of caspofungin and salts thereof containing about 0.25% weight by HPLC or less of caspofungin C₀ and salts thereof in the manufacture of a pharmaceutical composition for the treatment and prevention of infections caused by Pneumocystis carinii.

### DETAILED DESCRIPTION OF THE INVENTION

An analysis of commercial Cancidas® containing caspofungin diacetate shows that the commercial product (see example 6) contains at least 0.28% area by HPLC of caspofungin C₀ diacetate. The similarity in the structure of caspofungin and caspofungin C₀ makes their detection and separation from each other difficult.

The present invention provides pure caspofungin and salts thereof substantially free of caspofungin C₀and salts thereof. Also described are processes for preparing such pure caspofungin and salts thereof and an HPLC method that separates the two compounds and can be used to detect the level of caspofungin C₀ and its salts in caspofungin and its salts.

As used herein, the term “% weight by HPLC of caspofungin C₀ and salts thereof” refers to the weight of caspofungin C₀ and salts thereof in a sample of caspofungin and salts thereof as measured by HPLC using caspofungin as a reference standard, by conventional methods.

As used herein, the term “% weight by HPLC of pneumocandin C₀” refers to the weight of pneumocandin C₀ in a sample of pneumocandin B₀ as measured by HPLC using pneumocandin B₀ as a reference standard, by conventional methods.

In one embodiment, the present invention provides caspofungin and salts thereof of the following formula (n=0-3):

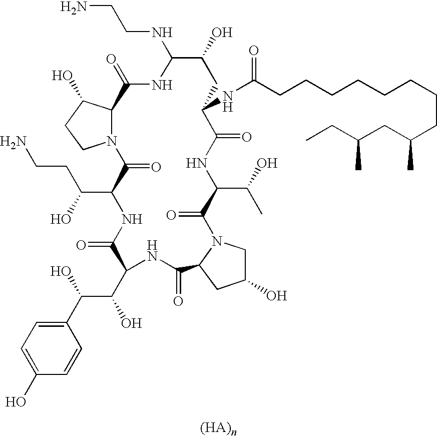

containing about 0.25% weight by HPLC or less of caspofungin C₀ and salts thereof of the following formula (n=1-3):

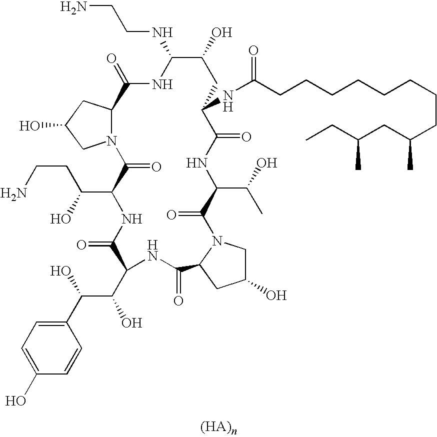

Preferably, HA in the above formula refers to: hydrochloric acid, hydrobromic acid, sulfuric acid, phosphoric acid, maleic acid, citric acid, tartaric acid, oxalic acid, ascorbic acid and acetic acid.

Preferably, HA is hydrochloric acid, acetic acid, or tartaric acid.

More preferably, the salt is a diacetate of the following formula:

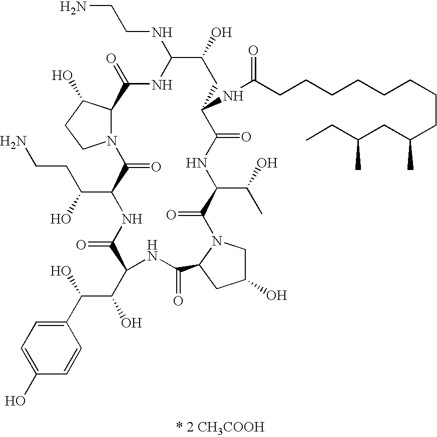

Preferably, caspofungin and salts thereof contain about 0.20% weight by HPLC or less, more preferably, about 0.15% weight by HPLC or less, even more preferably, about 0.10% weight by HPLC or less, and most preferably, about 0.05% weight by HPLC or less of caspofungin C₀ and salts thereof.

The level of caspofungin C₀ and salt thereof in caspofungin and salt thereof may be measured by an HPLC method comprising:
a) combining a sample comprising caspofungin or salt thereof in a mixture of ammonium phosphate buffer and methanol to obtain a solution;
b) injecting the solution to a silica gel based polyamine HILIC (hydrophilic interaction chromatography) column;
c) eluting the sample from the column using an eluent of a mixture of acetonitrile: isopropanol and ammonium phosphate buffer; and
d) measuring the content of Caspofungin C₀ using a UV detector;
   wherein the sample of Caspofungin in step a) may also contain Caspofungin C₀ or salt thereof.

Preferably, the column may be a YMC Polyamine II 150×4.6 mm, 5 μm column.

Preferably, the Column temperature may be 8° C.-12° C., most preferably 10° C.

Preferably the flow rate used in the HPLC method may be 0.9-1.1 ml/min, most preferably 1.0 ml/min.

Preferably, the pH of the buffer used in the HPLC method may be 3.0-4.0.

Preferably, the concentration of the buffer used in the HPLC method may be 0.018-0.022 M.

Preferably, the ratio of acetonitrile:isopropanol:ammonium phosphate buffer in the eluent may be 59:14:27-57:14:29 or 56:16:28-60:12:28, most preferably the ratio 58:14:28.

Preferably, the analysis of the amount of Caspofungin C₀ in the initial sample of Caspofungin may be performed using UV detection at 225 nm.

The above caspofungin and salt thereof may be prepared by a method comprising:
a) analyzing the level of pneumocandin C₀ of the following formula:

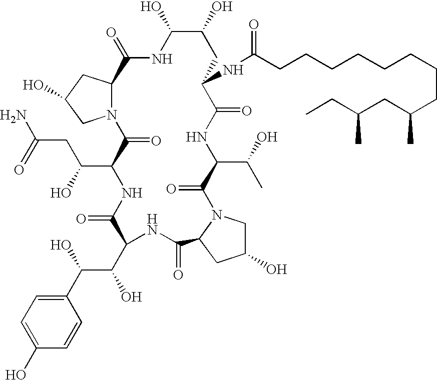

present in at least one initial sample of pneumocandin B₀ of the following formula:

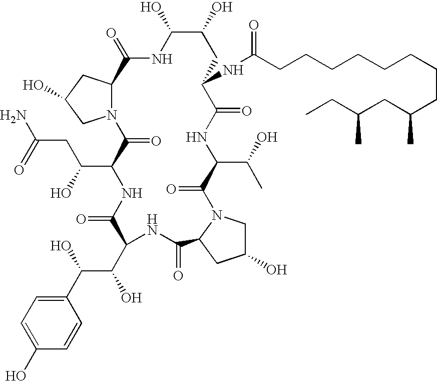

at least at a single stage during the synthesis process to control the amount of said caspofungin C₀ and salts thereof being present in the prepared caspofungin and salts thereof;
b) selecting as initial sample of pneumocandin B₀ that has no more than about 0.54% weight by HPLC of Pneumocandin C₀, and
c) preparing caspofungin and salts thereof containing about 0.25% weight by HPLC or less of caspofungin C₀ and salts thereof from the said initial sample of Pneumocandin B₀.

The analysis of the amount of Pneumocandin C₀ in the initial sample of pneumocandin B₀ may be performed using UV detection at 278 nm.

Preferably the initial sample of pneumocandin B₀ having no more than 0.54% weight by HPLC of pneumocandin C₀ is prepared by a process comprising the steps of:
a) purifying pneumocandin B₀ by chromatography; and
b) crystallizing the obtained pneumocandin B₀ from a solvent-antisolvent mixture.

Preferably, the solvent is a C₁-C₄ alcohol and the antisolvent is a C₃-C₇ ester.

Most preferably the solvent is methanol, and the anti-solvent is isopropyl acetate.

In one embodiment, the chromatography is carried out by dissolving pneumocandin in a suitable solvent such as methanol. Silica gel is added to the solution and the resulting mixture is evaporated, such as at a pressure of below one atmosphere, to prepare the loading charge. The loading charge can then be loaded silica gel on top of another column and connected. A proper eluent mixture, preferably ethyl acetate/methanol/water 84:9:7 solvent mixture, is used as an eluent. A suitable flow rate is 50 to 550 ml/h.

The fraction obtained from the column can be evaporated to oily residue, such as under reduced pressure (pressure of less than one atmosphere). Optionally, the oily residue can be diluted with a solvent and concentrated again to dryness under reduced pressure. Heating can be carried out at a temperature of 20° C. to about 65° C., such as about 60° C. The resulting solid/oil can be dissolved in a solvent to obtain a solution, followed by precipitation with an antisolvent. Preferably the solvent is a C₁-C₄ alcohol, more preferably methanol. Preferably the anti-solvent is a C₃-C₇ ester, more preferably isopropyl acetate.

The resulting precipitate can be recovered, such as by filtration. The precipitate can also be washed. It can be dried, such as by heating, preferably to a temperature of about 30° C. to about 50° C.

Crystallization is carried out in order to obtain a solid material for the caspofungin synthesis, since the solid pneumocandin B₀ is more stable than pneumocandin B₀ in solution.

Caspofungin can be prepared by reacting 4-Methoxyphenylthio-pneumocandin B₀ amine with ethylenediamine. The reaction can be carried out under dry conditions. The reaction can be cooled to about 15 to about 25° C. The reaction mixture can be stirred. Caspofungin is obtained and can be converted to a salt. To obtain a salt, the reaction mixture can be diluted with a C1 to C4 alcohol, such as methanol. An acid can then be added. In one embodiment acetic acid, preferably in mixture with water is added to the reaction mixture to obtain the diacetate salt.

The preparation of pneumocandin B₀ containing less than about 0.54% weight by HPLC of pneumocandin C₀ can be performed by the methods described in the prior art, such as the method in Journal of Antibiotics, 45, 1853 (1992), where the column chromatography which uses silica gel adsorbent together with EtOAc-MeOH-acetic acid (84:9:7) or dichloromethane-MeOH-water eluent, is repeated several times or by the method reported in examples 1 and 2.

The obtained caspofungin and salts thereof contain about 0.25% weight by HPLC or less of caspofungin C₀ and salts thereof. Preferably, the obtained caspofungin and salts thereof contain about 0.20% weight by HPLC or less, more preferably, about 0.15% weight by HPLC or less, even more preferably, about 0.1% weight by HPLC or less, and most preferably, about 0.05% weight by HPLC or less of caspofungin C₀ and salts thereof.

The preparation of caspofungin and salts thereof from pneumocandin B₀ can be performed for example, according to the process disclosed in example 4 herein.

The present invention further encompasses 1) a pharmaceutical composition comprising caspofungin and salts thereof containing about 0.25% weight by HPLC or less of caspofungin C₀ and salts thereof and optionally at least one pharmaceutically acceptable excipient, 2) the use of the above-described caspofungin and salts thereof containing about 0.25% weight by HPLC or less of caspofungin C₀ and salts thereof, in the manufacture of a pharmaceutical composition, wherein the pharmaceutical composition can be useful for the treatment of systemic fungal infections caused by Candida, Aspergillus, Histoplasma, Coccidioides and Blastomyces, and 3) the use of the above-described caspofungin and salts thereof containing about 0.25% weight by HPLC or less of caspofungin C₀ and salts thereof, in the manufacture of a pharmaceutical composition, wherein the pharmaceutical composition can be useful for the treatment and prevention of infections caused by Pneumocystis carinii.

The pharmaceutical composition can be prepared by a process comprising combining caspofungin and salts thereof containing about 0.25% weight by HPLC or less of caspofungin C₀ and salts thereof with at least one pharmaceutically acceptable excipient. The caspofungin and salts thereof containing about 0.25% weight by HPLC or less of caspofungin C₀ and salts thereof can be obtained by the process of the present invention as described above.

The caspofungin and salts thereof containing about 0.25% weight by HPLC or less of caspofungin C₀ and salts thereof of the present invention, particularly in a pharmaceutical composition and dosage form, can be used to treat systemic fungal infections caused by Candida, Aspergillus, Histoplasma, Coccidioides and Blastomyces and also treat and prevent infections caused by Pneumocystis carinii, in a mammal, preferably a human, comprising administering a treatment effective amount of the caspofungin and salts thereof containing about 0.25% weight by HPLC or less of caspofungin C₀ and salts thereof in the mammal. The treatment effective amount or proper dosage to be used can be determined by one of ordinary skill in the art, which can depend on the method of administration, the bioavailability, the age, sex, symptoms and health condition of the patient, and the severity of the disease to be treated, etc.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Analytical Method for Determination of Caspofungin C₀ Isomer in Caspofungin

| | |
|---|---|
| Column: | silica gel based polyamine HILIC column |
| | (150 × 4.6 mm, 5 μm) |
| Eluent: | acetonitrile:isopropanol:(0.025 M H₃PO₄ |
| | pH = 3.5/25% NH₃) 58:14:28 isocratic elution |
| Flow rate: | 1.0 ml/min |
| Column temperature: | 10° C. |
| Injected volume: | 20 μl |
| Run time: | 30 min |
| Sample: | ~500 μg/ml dissolved in buffer:methanol |
| | 2:8 mixture |

Typical chromatogram:

### Example 1

Preparation of Pneumocandin B₀ with Low Level of Pneumocandin C₀ (Scale-Up)

Pneumocandin starting material was purified by chromatography as described below. The starting material contained 68.54 area percent of pneumocandin B₀ and 1.80 area percent of pneumocandin C₀. The pneumocandin starting material was obtained from the conventional fermentation process as described in the references cited above, for example U.S. Pat. Nos. 5,194,377 and 5,202,309. An assay of the starting substance gave a purity of 48.73 percent by mass for pneumocandin B₀ and 1.28 percent by mass for pneumocandin C₀. Following the purification method of the example, the final product contained 0.54 weight percent of pneumocandin C₀. The purified substance gave a purity of 89.29 percent by mass.

### Chromatography Method

Silica gel 60 (0.015-0.040 mm) was used for the chromatography. Two chromatography columns (100 mm diameter, 230 mm column height, loaded with 500 g of silica gel and 100 mm diameter, 1000 mm column height, loaded with 4 kg silica gel) were prepared. The pneumocandin starting material in an amount of 100 g, where 48.73 g was active substance, was dissolved in 300 mL of methanol. The silica gel 60 (0.015-0.040 mm) in an amount of 250 g was added to the pneumocandin solution. This mixture was evaporated to dryness under reduced pressure to prepare the loading charge.

The loading charge was loaded as a layer on the top of the bed of 500 g silica gel. The columns were connected and eluted with an eluent of ethyl acetate/methanol/water 84:9:7 solvent mixture with a flow rate 550 ml/h. Fractions of 550 ml each were collected and several fractions were analyzed by HPLC using a UV detector of 278 nm.

Appropriate fractions were then combined.

Crystallization of the Main Fraction after Chromatography

The combined main fraction was evaporated to oily residue under reduced pressure. The oily residue was diluted with 500 ml of isobutanol, and concentrated again to dryness under reduced pressure. The heating temperature was approximately 60° C. The solid content was diluted with methanol to 198 g, and 1782 ml isopropyl acetate was added to the solution at ambient temperature to precipitate the purified material. After stirring for 60 minutes, the precipitates were filtered, and washed with 150 ml of isopropyl acetate. The washed precipitates were dried at 40° C. for 16 hours, providing a mass of dried substance of 35.2 g. The final product contained 0.54 weight percent of pneumocandin C₀, and gave a purity of 89.29 percent by mass for pneumocandin B₀.

Crystallization is carried out in order to obtain a solid material for the caspofungin synthesis, since the solid pneumocandin is more stable than pneumocandin in solution.

### Example 2

### Preparation of Pneumocandin B₀ Essentially Free of Pneumocandin C₀

Pneumocandin starting material was purified by chromatography. The starting material contained 72.57 area percent of pneumocandin B₀ and 2.13 area percent of pneumocandin C₀. An assay of the starting substance gave a purity of 62.36 percent by mass for pneumocandin B₀ and 1.81 percent by mass for pneumocandin C₀. Following the purification method of example 1, the final product contained 0 weight percent of pneumocandin C₀. The purified substance gave a purity of 99.08 percent by mass.

### Chromatography Step of the Purification Method

Silica gel 60 (0.015-0.040 mm) was used for the chromatography. Two chromatography columns (36 mm diameter, 230 mm column height, loaded with 50 g of silica gel and 36 mm diameter, 920 mm column height, loaded with 400 g silica gel) were prepared. The pneumocandin starting material in an amount of 10 g, where 6.236 g was active substance, was dissolved in 30 mL of methanol. The silica gel 60 (0.015-0.040, mm) in an amount of 25 g was added to the pneumocandin solution. This mixture was evaporated to dryness under reduced pressure to prepare the loading charge.

The loading charge was loaded as a layer on top of the bed of 50 g silica gel. The columns were connected and eluted with an eluent of ethyl acetate/methanol/water 84:9:7 solvent mixture with a flow rate 50 ml/h. Fractions of 50 ml each were collected and several fractions were analyzed by HPLC.

One fraction contained 99.44 area percent pneumocandin B₀, 0 area percent pneumocandin C₀.

Appropriate fractions were then combined.

Crystallization of Main Fraction after Chromatography

The combined main fraction contained 95.44 area percent pneumocandin B₀, 0 area percent pneumocandin C₀. The combined main fraction (1500 ml) was evaporated to oily residue under reduced pressure. The oily residue was diluted with 50 ml of isobutanol, and concentrated again to dryness under reduced pressure. The heating temperature was approximately 60° C. The solid content was diluted with methanol to ca. 15.1 g, and 135.9 ml isopropyl acetate was added to the solution at ambient temperature to precipitate purified material. After stirring for 60 minutes, the precipitates were filtered, and washed with 15 ml of isopropyl acetate. The washed precipitates were dried at 40° C. for 16 hours, providing a mass of dried substance of 3.81 g. The final product contained 0 weight percent of pneumocandin C₀, and gave a purity of 99.08 percent by mass for pneumocandin B₀.

### Example 3

### Preparation of Caspofungin C₀

### Step A:

Preparation of 4-methoxyphenylthio-pneumocandin C₀

Pneumocandin C₀ (1.2 g; assay: 34.9%; HPLC purity: 44.3 area %) was suspended in acetonitrile (25 ml) in a jacketed reactor fitted with thermometer, nitrogen inlet and mechanical stirrer.

The mixture was cooled to −15° C. by means of a thermostat, and 4-methoxythiophenol (0.25 g) was added in one portion. Trifluoroacetic acid (4.39 g) was added dropwise in about 15 min keeping the temperature between −10-−15° C.

The mixture was stirred at −15° C. for 22 h and quenched by the addition of water (75 ml) at a temperature below 0° C. The mixture was stirred at about 0° C. for 1 h then the precipitated solid was collected, washed twice with acetonitrile-water (1:3 v/v) (8 and 8 ml) and twice with acetonitrile (8 and 8 ml) to afford the crude product 0.98 g after drying in vacuum at less than 40° C. for 6 h.

The crude product was purified by silica gel column chromatography (36 g of silica gel; ethyl acetate-methanol (7:3 v/v) eluent) to afford a purified product, 0.69 g in the HPLC purity of 61.9 A%.

### Step B:

Preparation of 4-methoxyphenylthio-pneumocandin C₀ amine

4-Methoxyphenylthio-pneumocandin C₀ (0.68 g) was suspended in tetrahydrofuran (25 ml) then phenylboronic acid (0.065 g) was added, and the mixture was stirred at less than 40° C. until obtaining a solution (1 h).

A molecular sieve of 3 Å (5 g) was then added to the mixture and was allowed to stand at room temperature for about 16 h.

The molecular sieve was removed, washed with THF (2×5 ml) and the filtrate was charged to a four necked round bottomed flask fitted with nitrogen inlet, thermometer and a cooling bath.

The solution was cooled to −5° C. and borane-dimethylsulfide complex (0.36 g/90% pure/) was added and the mixture was stirred at 0-−5° C. for 6 h.

The reaction mixture was cooled to −15° C., and quenched by addition of 2N aqueous hydrochloric acid solution (1 ml).

The quenched mixture was stored in a freezer at −15° C. overnight, then was diluted with water (125 ml).

The diluted solution was filtered through a sintered glass filter and charged onto a 30 g reverse phase (LiChroprep RP-18,) column. The column was washed with acetonitrile-water (20:80 v/v; 180 ml) and the product was eluted with acetonitrile-water (40:60 v/v) by means of gravitation. Fractions of 20 ml each were collected and analyzed by TLC. The fractions showing the presence of the product were analyzed by HPLC.

The rich cuts (>85 A%) were combined, diluted with the same amount of water and charged to a the same column described above.

The product was eluted with methanol by means of gravitation, collecting 5×30 ml fraction which were analyzed by HPLC. The suitable fractions were combined and concentrated on a rotary evaporator at a temperature of less than 30° C. to afford 105 mg of the product in a HPLC purity of 94.7 A%.

### Step C:

### Preparation of Caspofungin C₀ Diacetate

4-Methoxyphenylthio-pneumocandin C₀ amine (100 mg) was added to ethylenediamine (0.5 ml) under nitrogen while stirring and cooling at room temperature.

The mixture was stirred at room temperature for 6 h then it was diluted with methanol (0.5 ml) while cooling with ice-water. The mixture of water (2 ml) and acetic acid (0.5 ml) was added under the same condition, and the mixture was diluted with water (7 ml). The solution was charged to 1.8 g reverse phase (2 pieces of ALTEC C18 cartridge) and the column was washed with the mixture of acetonitrile-water (20:80 v/v) and the product was eluted with acetonitrile-water (20:80 v/v+0.03% acetic acid). Fractions of 3 ml each were collected and analyzed by TLC. The fractions showing the presence of the product were diluted with the same amount of water. The solution was charged onto the same column described above, and the product was eluted with methanol collecting fractions of 2 ml which then were analyzed by TLC. The fractions containing the product were evaporated on a rotary evaporator at less than 30° C. to yield 50 mg (48%) of the product in the HPLC purity of 97.1 A%

### Example 4

Preparation of Caspofungin with a Low Content of Caspofungin C₀

One sample of pneumocandin B₀ purified by silica gel column chromatography according to example 1 was transformed to caspofungin according to the following examples.

### Step A:

Preparation of 4-methoxyphenylthio-pneumocandin B₀

Pneumocandin B₀ (I) (25.2 g) (assay: 89.3%; HPLC purity:91.0 A%; PC₀ content: 0.54%) was suspended in acetonitrile (630 ml) in a jacketed reactor fitted with thermometer, nitrogen inlet and mechanical stirrer.

The mixture was cooled to −15° C. by means of a thermostat, and 4-methoxythiophenol (5.88 g) was added in one portion. Trifluoroacetic acid (117.9 g) was added dropwise in about 20 min keeping the temperature between −10-−15° C.

The mixture was stirred at −15 C.° for 22 h and quenched by addition of water (1260 ml) at a temperature below 0° C. in about 60 min. The mixture was stirred at about 0° C. for 1 h then the precipitated solid was collected, washed twice with acetonitrile-water (1:3 v/v) (140 and 140 ml) and twice with acetonitrile (105 and 70 ml) to afford the product 23.97 g (85.2% ) after drying in vacuum at less than 40° C. for 24 h in the HPLC purity of 78.8 A% and assay of 72.2%.

### Step B:

Preparation of 4-methoxyphenylthio-pneumocandin B₀ amine

4-Methoxyphenylthio-pneumocandin B₀ (14.0 g) was suspended in tetrahydrofuran (500 ml) then phenylboronic acid (2.31 g) was added, and the mixture was stirred at less than 40° C. until obtaining a solution (4 h).

Molecular sieve of 3 Å (50 g) was then added to the mixture and was allowed to stand at room temperature for about 16 h to decrease the water content (LT 150 ppm).

The molecular sieve was removed, washed with THF (50 ml) and the filtrate was charged to a jacketed reactor fitted with nitrogen inlet, thermometer and a thermostat.

The solution was cooled to −5° C. and borane-dimethylsulfide complex (3.86 g/90 % pure/) was added in about 15 minutes at 0-−5° C. resulting in a dense gelatinous mixture in 30 min after addition which was stirred at about −5° C. for 10 h.

The reaction mixture was cooled to −15° C., and quenched by addition of 2N aqueous hydrochloric acid solution (8 ml) at −10-−15° C. in about 15 min resulting in a clear solution.

The quenched mixture was stored in a freezer at about −15° C. overnight, then was diluted with water (2200 ml).

The diluted solution was filtered through a sintered glass filter and charged onto a 295 g reverse phase (LiChroprep RP-18,) medium pressure column (36×460 mm) with the speed of about 18 ml/min. The column was washed with acetonitrile-water (20:80 v/v; 1800 ml; 18 ml/min) and the product was eluted with acetonitrile-water (40:60 v/v; about 14 ml/min). Fractions of 200 ml each were collected by means of a fraction collector and analyzed by TLC, than the fractions showing the presence of the product, by HPLC.

The rich cuts (>88 A%) were combined, diluted with water and charged to a 125 g of a reverse phase column (LiChroprep RP-18,).

The product was eluted with methanol by means of gravitation, collecting 5×120 ml fraction which were analyzed by HPLC. The suitable fractions were combined and concentrated on a rotary evaporator at a temperature of less than 30° C. and the product was precipitated by addition of acetonitrile.

The mixture was cooled to 2-8° C., the solid was collected, washed with acetonitrile (20 ml) and dried in a vacuum oven at room temperature for 24 h to yield 4.82 g (35.2%) of the product in a HPLC purity of 96.8 A% and assay of 91.9%.

### Step C:

### Preparation of Caspofungin Diacetate

4-Methoxyphenylthio-pneumocandin B₀ amine (4.34 g) was added to ethylenediamine (18.5 ml) under nitrogen while stirring and cooling at 15-25° C.

The mixture was stirred at room temperature for 6 h then it was diluted with methanol (24 ml) while cooling with ice-water at 15-25° C. The mixture of water (90 ml) and acetic acid (24 ml) was added under the same condition, and finally, the pH of the mixture was adjusted to 6-7 by addition of acetic acid (8 ml).

The neutralized mixture was diluted with water (310 ml), washed with toluene (3×47 ml) and filtered through a G-4 sintered glass filter. The solution was charged to a 300 g reverse phase (SP-100-15-ODS-P; Daiso Co. Ltd.) medium pressure column (36×460 mm) with the speed of about 14 ml/min, and the product was eluted with acetonitrile-water (20:80 v/v+0.01% acetic acid; 14 ml/min). Fractions of 100 ml each were collected and analyzed by TLC, then the fractions showing the presence of caspofugin, by HPLC.

The rich cuts (>99.0 A% (HPLC) were combined and lyophilized to afford 3.19 g (71.7%) caspofungin acetate as a cotton-like white solid.

The Caspofungin C₀ content of the product was 0.22% on the basis of HPLC analysis.

### Example 5 (Prophetic Example)

### Preparation of Caspofungin Essentially Free of Impurity Caspofungin C₀

Starting from 3.2 g of the product of Example 2, following the procedure described in Example 4, 0.40 g of caspofugin diacetate being free of caspofungin C₀ are expected to be obtained.

### Example 6: Analysis of Cancidas® (Comparative Example)

US samples of Cancidas® were analyzed prior to the expiry date, using the above HPLC method:

| Batch. No. | Expiry. date | C₀ content, % area by HPLC |
|---|---|---|
| 1073R | September 2007, | 0.30 |
| 0286F | December 2007, | 0.28 |
| 0130F | November 2007, | 0.29 |
| 0288F | January 2008, | 0.29 |
| 0827900 | December 2008, | 0.34 |
| 0836170 | May 2009, | 0.47 |

The samples were prepared according to the provided method. Lyophilized material was dissolved in the diluent to obtain a solution containing about 500 μg/ml Caspofungin, and was then injected into the HPLC.

1. Caspofungin and salts thereof of the following formula (n=0-3) 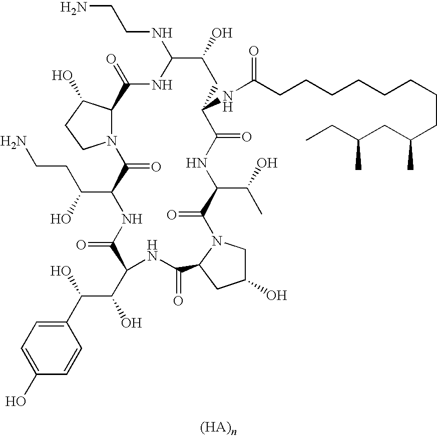
containing about 0.25% weight by HPLC or less of Caspofungin C₀ and salts thereof of the following formula (n=0-3):

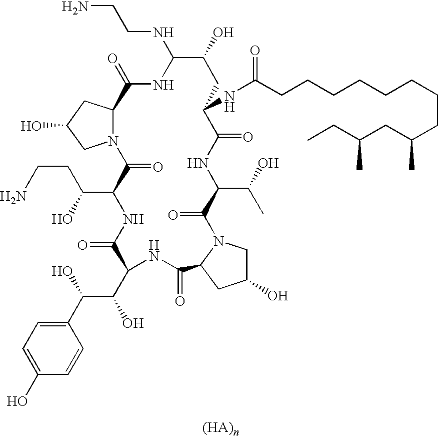

2. The caspofungin and salts thereof of claim 1 containing about 0.20% weight by HPLC or less of Caspofungin C₀ and salts thereof.

3. The caspofungin and salts thereof of claim 2 containing about 0.15% weight by HPLC or less of Caspofungin C₀ and salts thereof.

4. The caspofungin and salts thereof of claim 3 containing about 0.10% weight by HPLC or less of Caspofungin C₀ and salts thereof.

5. The caspofungin and salts thereof of claim 4 containing about 0.05% weight by HPLC or less of Caspofungin C₀ and salts thereof.

6. The Caspofungin and salts thereof of claim 1, wherein HA is an acid selected from the group consisting of: hydrochloric, hydrobromic, sulfuric, phosphoric, maleic, citric, tartaric, oxalic, ascorbic and acetic acids.

7. The Caspofungin and salts thereof of claim 6 wherein HA is an acid selected from the group consisting of hydrochloric, acetic, or tartaric acid.

8. The Caspofungin and salts thereof of claim 7 wherein the salt is a diacetate salt of the following formula:

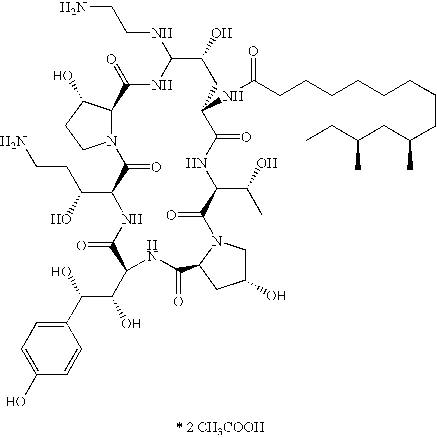

9. A process for preparing Caspofungin and salts thereof as defined in claim 1 from an initial sample of Pneumocandin B₀ comprising:
a) analyzing the level of Pneumocandin C₀ of the following formula: 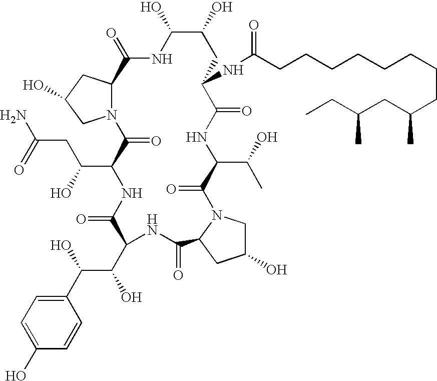
present in at least one initial sample of Pneumocandin B₀ of the following formula: 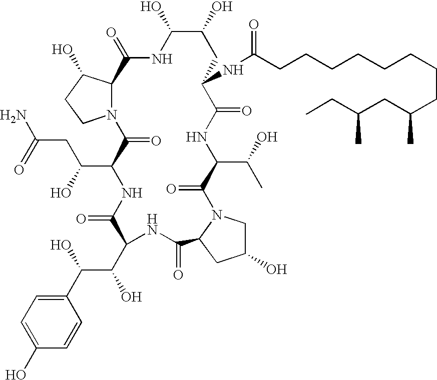
at least at a single stage during the synthesis process to control the amount of said Caspofungin C₀ and salts thereof being present in the prepared caspofungin and salts thereof;
b) selecting as initial sample of Pneumocandin B₀ that has no more than about 0.54% weight by HPLC of Pneumocandin C₀, and
c) preparing caspofungin and salts thereof containing about 0.25% weight by HPLC or less of Caspofungin C₀ and salts thereof from the said initial sample of Pneumocandin B₀.

10. The process of claim 9 wherein the Pneumocandin B₀ that has no more than about 0.54% weight by HPLC of Pneumocandin C₀ is prepared according to a process comprising the steps of:
a) purifying Pneumocandin B₀ by chromatography; and
b) crystallizing the obtained Pneumocandin B₀ from a solvent-antisolvent mixture;
wherein the solvent is a C₁-C₄ alcohol and the antisolvent is a C₃-C₇ ester.

11. The process of claim 9 wherein analysis of Pneumocandin C₀ in the initial sample of Pneumocandin B₀ is performed using UV detection at 278 nm.

12. The process of claim 10 wherein the solvent is methanol and the antisolvent is isopropyl acetate.

13. The process of claim 10 wherein chromatography is carried out by a process comprising the steps of:
a) dissolving pneumocandin B₀ in a suitable solvent to obtain a solution;
b) adding silica gel to the solution;
c) evaporating the solution to prepare the loading charge;
d) loading the loading charge to silica gel on top of a column;
e) eluting the column using a proper eluent mixture at a suitable flow rate to obtain a fraction; and
f) recovering the fraction.

14. The process of claim 13 wherein the suitable solvent in step a) is methanol.

15. The process of claim 13 wherein the suitable eluent mixture in step e) is ethyl acetate/methanol/water 84:9:7.

16. The process of claim 13 wherein the suitable flow rate in step e) is 50 to 550 ml/h.

17. The process of claim 16 wherein the suitable flow rate in step e) is 550 ml/h.

18. The process of claim 13 wherein the fraction of step f) is recovered by evaporation under reduced pressure of less than one atmosphere.

19. The process of claim 13 wherein the fraction of step f) is recovered by diluting the fraction in a solvent and concentrating again to dryness under reduced pressure.

20. The process of claim 18 wherein heating is carried out at a temperature of 20° C. to about 65° C.

21. The process of claim 13 wherein the obtained precipitate from step b) is recovered by filtration.

22. The process of claim 13 wherein the obtained precipitate from step b) is washed.

23. The process of claim 13 wherein the obtained precipitate from step b) is dried by heating at a temperature of about 30° C. to about 50° C.

24. A process for measuring the level of caspofungin C₀ and salts thereof in a sample of caspofungin and salts comprising the steps:
a) combining a sample comprising of caspofungin or salt thereof in a mixture of ammonium phosphate buffer and methanol to obtain a solution;
b) injecting the solution to a silica gel based polyamine HILIC (hydrophilic interaction chromatography) column;
c) eluting the sample from the column using an eluent of a mixture of acetonitrile: isopropanol and ammonium phosphate buffer; and
d) measuring the content of Caspofungin C₀ using a UV detector.

25. The process of claim 24 wherein the silica gel based polyamine HILIC (hydrophilic interaction chromatography) column is a YMC Polyamine II 150×4.6 mm, 5 μm column.

26. The process of claim 24 wherein the Column temperature used in the HPLC method is 8° C.-12° C.

27. The column of claim 26 wherein the Column temperature used in the HPLC method is 10° C.

28. The process of claim 24 wherein the flow rate used in the HPLC method is 0.9-1.1 ml/min.

29. The process of claim 28 wherein the flow rate used in the HPLC method is 1.0 ml/min.

30. The process of claim 24 wherein the pH of the buffer used in the HPLC method is 3.0-4.0.

31. The process of claim 24 wherein the buffer concentration used in the HPLC method is 0.018-0.022 M.

32. The process of claim 24 wherein the ratio of acetonitrile:isopropanol:ammonium phosphate buffer in the eluent is 59:14:27-57:14:29 or 56:16:28-60:12:28 (v/v).

33. The process of claim 32 wherein the ratio of acetonitrile:isopropanol:ammonium phosphate buffer in the eluent is 58:14:28 (v/v).

34. The process of claim 24 wherein the a UV detection at 225 nm is used.

35. A pharmaceutical composition comprising Caspofungin and salts thereof, as defined in claim 1, and at least one pharmaceutically acceptable excipient.

36. A method of treatment comprising administering to a mammal in need thereof a pharmaceutical composition comprising Caspofungin and salts thereof, as defined in claim 1, for the treatment of systemic fungal infections caused by Candida, Aspergillus, Histoplasma, Coccidioides and Blastomyces.

37. A method of treatment comprising administering to a mammal in need thereof a pharmaceutical composition comprising Caspofungin and salts thereof, as defined in claim 1, for the treatment and prevention of infections caused by Pneumocystis carinii.
